(19) United States
(12) Patent Application Publication
(10) Pub. No.: US 2009/0240138 A1
(43) Pub. Date: Sep. 24, 2009

(54) Diffuse Optical Tomography System and Method of Use

(76) Inventor: Steven Yi, Vienna, VA (US)

Correspondence Address: STEVEN L. NICHOLS;RADER, FISHMAN & GRAUER PLLC, 10653 S. RIVER FRONT PARKWAY, SUITE 150, SOUTH JORDAN, UT, 84095, US

(21) Appl. No.: 12/050,733

(22) Filed: Mar. 18, 2008

(51) Int. Cl. A61B 6/03

(52) U.S. Cl. 600/425

(57) ABSTRACT

A multi-spectrum diffuse optical tomography imaging system for in vivo non-contact imaging includes an illumination source assembly for illuminating a specimen; a first filter wheel adapted to control an intensity of illumination directed onto the specimen; a three-dimensional (3D) imaging assembly for outputting an electronic (3D) model of the specimen; and a sensor assembly for capturing a response of the specimen to the illumination source assembly and outputting corresponding tomography data. The system combines the tomography data and the 3D model for the specimen.

### BACKGROUND

Diffuse Optical Tomography (DOT) and Fluorescence Molecular Tomography (FMT) are implementations of optical imaging that three-dimensionally resolve and quantify the bio-distribution of chromophores and fluorescence reporters, respectively, through several millimeters to centimeters of tissue. With this ability comes the capacity to resolve various bio-markers and study disease evolution and the effects of treatment. DOT and FMT may also be used to measure physiological parameters, such as (1) oxygen saturation of hemoglobin and blood flow based on intrinsic tissue contrast, (2) molecular tissue function, and (3) gene-expression based on extrinsically administered fluorescent probes and beams. DOT and FMT offer several advantages over existing radiological techniques, such as being non-invasive and non-ionizing.

DOT imaging includes illuminating the tissue with a light source and measuring the light leaving the tissue with a sensor. A model of light propagation in the tissue is developed and parameterized in terms of the unknown scattering and/or absorption as a function of position in the tissue. Then, using the model together with the ensemble of images over all the sources, the DOT imaging system inverts the propagation model to recover the scatter and absorption parameters.

A DOT image is actually a quantified map of optical properties and can be used for quantitative three-dimensional imaging of intrinsic and extrinsic adsorption and scattering, as well as fluorophore lifetime and concentration in diffuse media such as tissue. These fundamental quantities can then be used to derive tissue oxy- and deoxy-hemoglobin concentrations, blood oxygen saturation, contract agent uptake, and organelle concentration. Such contrast mechanisms are important for practical applications such as the measurement of tissue metabolic activity, angiogenesis and permeability for cancer detection as well as characterizing molecular function.

A typical DOT system uses lasers so that specific chromophores are targeted and the forward model is calculated for the specific wavelengths used. Laser diodes have been customarily used as light sources since they produce adequate power and are stable and economical. Light is usually directed to and from tissue using fiber optic guides since this allows flexibility in the geometrical set-up used. For optical coupling, the fibers must be in contact with tissue or a matching fluid. Use of a matching fluid helps to eliminate reflections due to mismatches between indices of refraction between silica, air, and tissue.

Advanced DOT algorithms require good knowledge of the boundary geometry of the diffuse medium imaged in order to provide accurate forward models of light propagation within this medium. A forward model is a representation of the representative characteristics of the volume being studied. Currently, these boundary geometries are forced into simple, well known shapes such as cylinders, circles, or slabs. In addition to not accurately representing the shape of the specimen to be analyzed, the use of these shapes forces the specimen being analyzed to be physically coupled to the shape either directly or by the use of a matching fluid as discussed above.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is a diagram showing one illustrative embodiment of an improved diffuse optical tomography system, according to principles described herein.

FIG. 2 is a diagram showing one illustrative embodiment of an illumination source assembly and a linear motion stage for directing illumination onto a specimen, according to principles described herein.

FIG. 3 is a diagram showing one illustrative method of using a diffuse optical tomography system, according to principles described herein.

FIG. 4 is a diagram showing one illustrative embodiment of a spectrum source in combination with a three-dimensional (3D) camera assembly, according to principles described herein.

FIG. 5 is a diagram showing a table containing transmittance values for a plurality of neutral density filters used in one illustrative embodiment, according to principles described herein.

FIG. 6 is a diagram showing an axial view of a FMT image slice of a specimen acquired using one illustrative embodiment, according to principles described herein.

FIG. 7 is a diagram showing an illustrative reconstructed three-dimensional specimen using a diffuse optical tomography system, according to principles described herein.

Throughout the drawings, identical reference numbers designate similar, but not necessarily identical, elements.

### DETAILED DESCRIPTION

An improved diffuse optical tomography system for in vivo, non-contact imaging includes an illumination source for illuminating a specimen, a spectrum source for projecting a spectrum of visible light onto the specimen in addition to the illumination from the illumination source, at least one sensor configured to capture the response of the specimen to the illumination and to the projection of the spectrum. The improved diffuse optical tomography system rapidly captures three-dimensional boundary geometry of, and the corresponding diffuse optical tomography measurements of, a specimen.

As used herein and in the appended claims, the term “tomography data” will be used to refer to data that is produced, as described above, from Diffuse Optical Tomography or Fluorescence Molecular Tomography. Consequently, “tomography data” refers to data produced by passing light or other electromagnetic radiation through a tissue specimen and then recording and interpreting the resulting transmission and scattering of the light or other radiation by the specimen. As used herein, “specimen” shall be broadly understood to mean any volume or surface to be analyzed.

As used herein and in the appended claims, the term “pixel” shall be broadly understood to mean an individual element of a picture or the hardware used to produce or represent an individual picture element. As used herein and in the appended claims, the term “voxel” shall be broadly understood to mean any element of a three-dimensional or volumetric model, display or representation of a specimen or other three-dimensional body.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

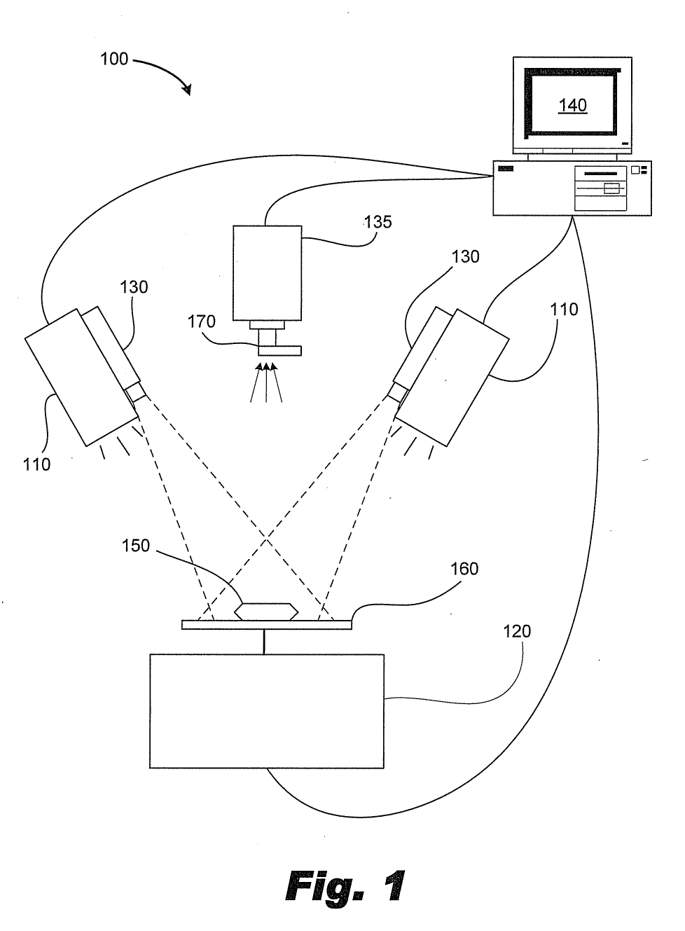

FIG. 1 illustrates an improved diffuse optical tomography (DOT) system (100) that comprises at least one spectrum source assembly (110), an illumination assembly (120), at least one three-dimensional (3D) camera assembly (130), and a sensor assembly (135), all coupled to a processing device such as computer (140). The system (100), with these components, is able to generate a 3D model or surface profile for the specimen (150) and then seamlessly and instantly associate tomography data with the 3D model. Consequently, the system provides a very accurate 3D model or framework in which tomography data is accurately placed. Each of the system components will be described in more detail below.

In the illustrated embodiment, the system (100) includes at least two spectrum source assemblies (110) and corresponding 3D camera assemblies (130). The spectrum source assemblies (110) project a spectrum or rainbow of visible or other light onto the surface of a specimen (150) resting on a positioning plate (160). As will be described herein, the spectrum source assemblies (110) and the corresponding 3D camera assemblies (130) produce the 3D model or surface profile for the specimen (150). In the illustrated example, two spectrum source assemblies (110) and corresponding 3D camera assemblies (130) are used to produce a complete 3D model of the surface of both sides of the specimen. For example, the spectrum source assembly (110) and corresponding 3D camera assembly (130) of the right of FIG. 1 can observe and model portions of the specimen surface that are out of the line of sight of the other spectrum source assembly and corresponding 3D camera assembly, and vice versa.

The illumination assembly (120) projects light onto and through the specimen (150). As shown in FIG. 1, the illumination assembly (120) is located on the opposite side of the specimen (150) from the sensor assembly (135). The illumination assembly (120) and sensor assembly (135) are used to produce the desired tomography data regarding the specimen. As noted above, this tomography data is registered and placed electronically within the 3D model of the specimen (150) produced by the 3D camera assemblies (130). The sensor assembly comprises a sensor (135), such as a near-infrared (NIR) camera, and a wavelength filter wheel (170) adapted to control the wavelength of the response captured by the sensor (135).

This data from the sensor assembly (135) and the 3D camera assemblies (130) are output to the computer (140). The computer (140) processes the data derived from the sensor assembly (135) and the 3D camera assemblies (130) to produce the desired tomography data regarding the specimen that is registered and placed electronically within a 3D model of the specimen (150). The computer then displays the results on a display device attached to the computer, such as a monitor.

Any 3D camera assemblies and DOT sensor assembly may be used to capture the specimen responses. One example of a suitable 3D camera assembly (130) is one that is configured to be used with a spectrum source assembly (110) as in the embodiment shown in FIG. 1. However, other suitable 3D camera assemblies include laser-scanning systems.

In the example shown in FIG. 1, the spectrum source assembly (110) is configured to project a spectrum of light of spatially varying wavelengths in the visible range onto the specimen (150). The response of the specimen to the applied light may be used to determine three-dimensional boundary conditions of the specimen. The three-dimensional boundary may be determined by utilizing triangulation. A triangle is uniquely formed by the distance between the spectrum source assembly (110), the 3D camera assembly (130), and the point on the specimen (150). The spectrum source assembly (110) may include a linear variable wavelength filter (LVWF, not shown). Light projected from the spectrum source assembly through the LVWF falls onto the specimen (150) as a rainbow spectrum. The wavelength of the coated color of the LVWF in a specific location is linearly proportional to the displacement of the location from the LVWF's blue edge. Accordingly, the specific pixel characteristics at each point constrain the system, thereby providing accurate information about the three-dimensional location of the point. See, e.g., U.S. Pat. No. 6,147,760 to Geng, which is incorporated herein by reference in its entirety.

Referring again to the sensor assembly (135), the wavelength filter wheel (170) may comprise a plurality of filters which allow the assembly (135) to switch between spectrums or wavelength bands. Depending on the wavelength of light to be detected, the filter wheel (170) is rotated to position a different filter over the pupil of the sensor (135). Consequently, the sensor (134) may tuned to, and capture, different wavelengths from which tomography data can be produced. The wavelength filter wheel (170) may include a laser filter and a fluorescence filter. The laser filter may be used to examine chromophores in the specimen, while the fluorescence filter may be used to resolve and quantify fluorescence reporters (fluorophores, which cause molecules in the specimen to be fluorescent) in the specimen. The computer may control the rotation of the wavelength filter wheel (170) such that the specimen of both filters may be simultaneously monitored.

The illumination source assembly (120) is configured to apply light in, for example, the visible spectrum to the specimen (150). As indicated above, the response of the specimen (150) to the applied light from the illumination source assembly may be used to determine internal characteristics of the specimen (150) such as the spectroscopic information about the biochemical structure of a tissue specimen. The information about the biochemical structure, the tomography data, is obtained by capturing and processing the specimen's response to illumination by the illumination source assembly (120). The spectroscopic information may reveal physiological parameters (e.g., oxygen saturation of hemoglobin and blood flow) based on intrinsic tissue contrast, molecular tissue function, as well as gene-expression based on extrinsically administered fluorescent probes and/or beacons.

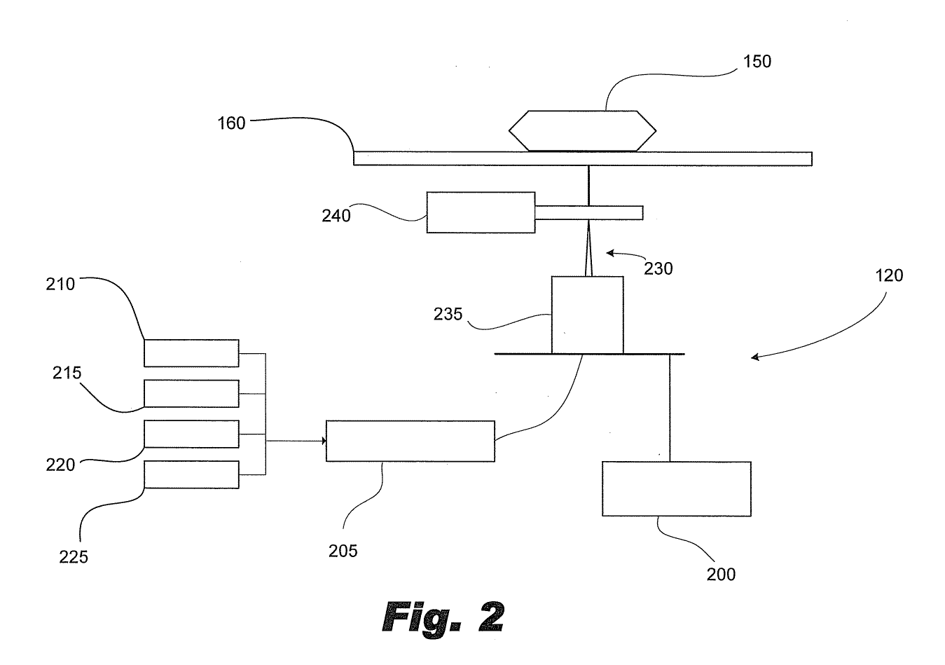

FIG. 2 is a schematic view of an exemplary embodiment of an illumination source assembly (120) and a linear motion stage (200). This illumination assembly (120) generates light within the spectral “window” for DOT imaging of soft tissue, which is between about 650 nm and 850 nm.

The illumination assembly (120) may include a beam combiner (205) to combine light generated by a number of laser sources (210, 215, 220, 225), each of which produces a beam of different wavelength. These beams are combined into a single composite beam (230) by the use of beam combiner (205). In this manner, the response of the specimen to the illumination source assembly (120) includes the specimen's response to light comprising a plurality of wavelengths. The beam combiner (205) may comprise coated high-pass wavelength filters with transmission and reflectance properties selected to facilitate formation of the composite beam (230). The composite beam (230) is directed from the beam combiner (205), using fiber optics for example, onto a location on the specimen (150). The composite beam (230), after exciting an optical filter, may be focused on the specimen with its use, for example, of a focus lens (235). A linear motion stage (200) may be used to reposition the beam (230) relative to the specimen.

The illumination source assembly (120) also comprises a neutral density (ND) filter wheel (240) designed to control the intensity of the composite beam directed onto the specimen (150). The ND filter wheel comprises a number of different filters, five, for example, that attenuate the power of the lasers to different degrees. Consequently, the filter wheel (240) can be rotated to bring different filters into optical coupling with the lens (235) and the composite beam (230) to selectively attenuate the beam (230). Consequently, the filter wheel can provide flexibility in producing different levels of laser power. For example, the various filters may provide transmittance factors of, respectively 25.12%, 50.12%, 63.10% and 79.43%. A filter with a lower transmittance percentage is used when the NIR images received by the sensor (135, FIG. 1) are too bright.

The composite beam (230) may be directed to any location on the specimen (150). The location on the specimen (150) to which the composite beam (230) is directed may be controlled, for example, by the linear motion stage (200). The linear motion stage may be capable of moving along a plurality of axes parallel to the positioning plate (160) on which the specimen (150) rests, preferable along two axes, each perpendicular to the other. The operation of the motorized linear motion stage (200) may be controlled by the computer (140; FIG. 1). This arrangement allows the light source configuration to be changed in real-time or “on the fly,” thereby facilitating the development of customized illumination plans for specific types of specimens, without changing the system hardware.

Accordingly, the illumination source assembly (120) may be a multi-wavelength composite source that integrates multiple spectral light sources, such as the lasers (210, 215, 220, 225), into a single beam (230) and positions the multi-spectral, point-light spot onto any location on the surface of the specimen. This configuration also allows for the illumination source assembly (120) to control the intensity of the light directed onto the specimen (150) because the ND filter wheel (240) may change filters in real-time to change the attenuation of the beam (230). Consequently, the response captured by the sensor assembly can be prevented from saturating the sensor (135) when the beam (230) is illuminating brighter areas of the specimen (150). Conversely, the illumination source assembly (120) may increase in brightness and power by adjusting the position of the ND filter wheel (240) to adequately illuminate darker areas of the specimen (150). The ND filter wheel (240) may be controlled by the computer (140) in accordance with the response captured by the sensor (135).

The ability of a DOT system to image fluorophores associated with molecular functions allows, for example, the three-dimensional imaging and quantifying of the up-regulation of cathepsin B in murine tumor models. Photon detection has high intrinsic sensitivity allowing the detection of fluorescent molecules at nano- to pico-molar concentrations, similar to isotope concentrations in nuclear medicine. Fluorescent molecules may be quenched and de-quenched, enabling the design of molecular switches or ‘beacons”. The probes are essentially non-fluorescent in their native (quenched) state and become highly fluorescent after enzyme-mediated release of fluorophores, resulting in signal amplification of up to several hundred-fold, depending on the specific design. The use of multiple probes allows a single enzyme to cleave multiple fluorophores, thus resulting in one form of signal amplification. Further, the reduction of background “noise” by several orders of magnitude is possible because very specific enzyme activities can potentially be interrogated while multiple probes can be arranged on delivery systems to simultaneously probe for a spectrum of enzymes.

Operation in intrinsic mode provides for characterization of internal processes as discussed above. Operation in fluorescence mode can direct the selection of wavelengths that are appropriate for excitation of the available dyes. The lasers (210, 215, 220, 225) may be pulsed lasers with variable duty cycles. In pulsed mode, the lasers (210, 215, 220, 225) may inject 100 megawatts (mW) of laser light onto the specimen for spectroscopic purposes. Alternatively, the lasers (210, 215, 220, 225) may produce constant wattage beams of less than 50 mW. The lasers may be laser diodes as monochromic light sources because they are less expensive and easier to operate than other types of lasers. Accordingly, the present system allows for selection between characterizations of intrinsic or extrinsic characteristics as determined by constant or pulsed light conditions.

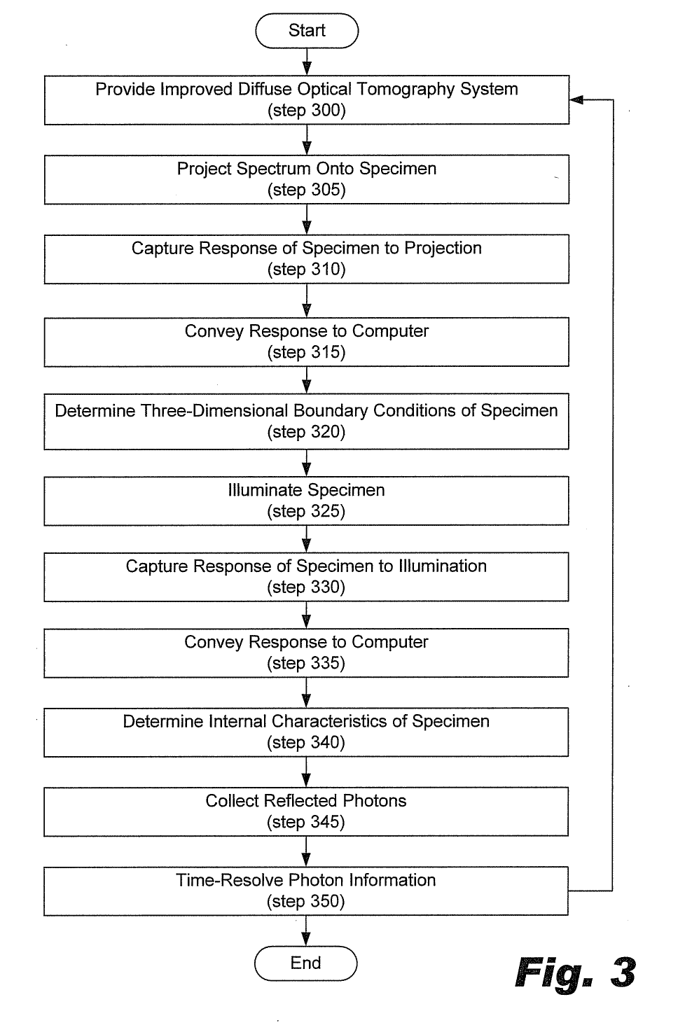

FIG. 3 is a flowchart illustrating a method of using the diffuse optical tomography system described herein. The method begins by providing the described diffuse optical tomography system (step 300). The diffuse optical tomography system generally includes an illumination source assembly, a spectrum source assembly, at least one 3D camera assemblies, a sensor assembly, and a computer.

The computer directs the spectrum source assembly to project at least one spectrum onto a specimen (step 305). The spectrum may be a spectrum of spatially varying wavelengths, such as that produced by an LED-based pattern projector or an LVWF. The response of the specimen to the projected spectrum is then captured by the 3D camera assemblies (step 310) and conveyed to the computer (step 315).

The computer processes the spectrum projection data in order determine the three-dimensional boundary conditions of the specimen (step 320). U.S. Pat. No. 5,675,407 to Geng, issued Oct. 7, 1997, describes a novel three-dimensional surface profile measuring technique that is able to acquire full frame, dynamic 3D images of objects with complex surface geometries at high speed. This patent is incorporated by reference in its entirety.

After or simultaneously with acquiring and processing the three-dimensional boundary conditions (step 320), the computer directs the illumination source assembly to illuminate the specimen (step 325). The response of the specimen to the illumination is captured by the sensor assembly (step 330) and conveyed to the computer (step 335). The computer processes the illumination data in order to determine the internal characteristics of the specimen in the area of the illumination (step 340), i.e., to produce tomography data.

This processing may include processing calculation of a forward problem and an inverse problem. The forward problem is defined to predict the light propagation pattern traveling through scattering medium (such as human tissues), given the optical properties of the medium. The inverse problem is then set to estimate the tissue optical properties (reconstruct the images) based on the optical intensity distribution measured along the surface of the speciment. Unlike X-Ray photons that travel in virtually straight paths, optical photons traveling into tissue experience significant scattering form the cellular structures (mitochondria, other organelles and cellular membranes) and follow “diffuse” propagation patterns.

Due to their diffuse pattern of propagation, near-infrared (NIR) photons produced by the illumination source assembly probe “volumes” and not “slices” as in other medical imaging techniques. Therefore tomographic techniques using real measurements of NIR photons reveal volumetric information and should be constructed as three-dimensional problems to better describe the underlying medium with improved accuracy. Efficient solutions to the forward problem exist by means of the diffusion approximation to the radiative transport or Boltzmann transport equation. The goal of DOT imaging is to reconstruct a spatial map of the optical contrast from fluence measurements at the boundary of the tissue under investigation. This is known as the “inverse problem.” From these maps, other biological characteristics, such as maps of blood volume, oxygen concentration or gene-expression can be derived. The general idea for solving the inverse problem is to use an accurate forward problem that best predicts the photon propagation into the medium under investigation and compare its findings with the actual measurements in an iterative fashion that minimizes the difference between the fluence measurements and the forward model outputs.

Intrinsic tissue contrast may be captured that distinguishes between cancers and benign/normal lesions based on optical absorption and related to angiogenesis, apoptosis, necrosis, and hyper-metabolism; and optical scattering related to the size and concentration of cellular organelles. Extrinsic tissue contrast may be captured with the ability to resolve with high sensitivity gene-expression and molecular signatures in-vivo by resolving and quantifying novel classes of fluorophores that probe specific molecular targets, such as cellular receptors, enzymes/proteins and nucleic acids.

DOT fundamentally produces quantitative images of intrinsic and extrinsic absorption, scattering and fluorescence as well as fluorophore concentration and lifetime in diffuse media such as tissue. These fundamental quantities can then be used to derive tissue oxy- and deoxyhemoglobin concentrations, blood oxygen saturation, contrast agent uptake, fluorophore activation and organelle concentration. Such novel contrast mechanisms are important for practical applications such as the measurement of tissue metabolic state, angiogenesis, and permeability for cancer detection, the measurement of functional activity in brain and muscle, the detection of hematomas and the elucidation of molecular pathways.

Of particular importance are novel advances in fluorescent molecular probes and the ability to detect them in-vivo using fluorescence-mediated molecular tomography. The technology is a special application of DOT to molecular fluorescent probes and beacons and is explained in more detail due to the significant impact it may have into clinical research. The use of light in the visible spectrum is non-ionizing (photon energy is ˜2 eV) and is substantially harmless in small doses. Accordingly, the use of light in the visible spectrum can be used for regular screening as well as continuous monitoring. Further, the use of visible light in the present system is non-invasive in that it does not require physical coupling between the light sources and the specimen. In addition, devices using visible light are relatively inexpensive and portable as compared to x-ray computed tomography (CT) and magnetic resonance imaging (MRI) and therefore could be used in emergency room applications, as well as for continuous bedside monitoring.

The accurate characterization of tissue spectral optical properties is important for accurate DOT inversions. The background optical properties may be measured on a per animal basis using time-resolved technology for the characterization of the average background absorption and reduced scattering coefficient. Light emanating 3-10 mm away from the impinging light source may be collected through fiber bundles that are directed to a time-correlated diffuse spectrometer (reflectometer) (step 345). Multiple-distance measurements allow probing of tissues at different depths to allow for a more representative average value for tissue of the specimen examined.

A photo-multiplier tube collects photons arriving after diffusing into tissue due to injected photon pulses with ˜100 picosecond widths at very low powers. The detected photons generate electrical pulses at the output of the photon sensor that are subsequently time-resolved using a multi-channel analyzer unit (step 350). While photons propagating for source-sensor large separations could be detected by a standard photo-multiplier tube (PMT) for detection, herein it may be desirable to use a multi-channel-plate (MCP) due to the low separations proposed. A multi-channel plate allows for minimum photon-detection temporal blurring leading to small quantification errors. This is particularly important in this implementation due to the relative source-sensor proximity that yields relative short propagation times of photons in tissue.

The detected data (called time resolved curves) are fitted to a Kirchoff Approximation solution of the diffusion equation for the forward problem using the geometry detected from the 3D camera assemblies, assuming a flat surface for the central points at which the specimen makes contact with the positioning plate. Multiple detection points can be implemented using time-sharing of the fiber bundle outputs onto the same sensor. Spectral information can be simultaneously acquired (up to 8 wavelengths) using appropriate time delays between the laser photon sources.

In summary, the present method provides for rapid and near simultaneous acquisition of accurate three-dimensional boundary and tomographic data. This process may be repeated at as many points as necessary in order to obtain an appropriately detailed image.

To facilitate the improvement of developments in free-space DOT/FMT imaging performance, it is important to retrieve the three-dimensional surface and a common coordinate system for the illumination system, the detection system and the specimen. To improve this process, a 3D-DOT algorithm based on a silhouette-based 3D surface reconstruction method may be developed into a 360 degree free-space system. For tomographic reconstructions of the chromophores/fluorescence distribution, the normalized Born approximation may be employed, which utilizes a synthetic measurement generated as the ratio of the measured chromophores/fluorophores perturbation or intensity to the corresponding measured intensity at the excitation wavelength for each spectrum/illumination source position and the detector positions.

A forward model may be generated to predict photon propagation in a diffuse medium. However, to accommodate for non-contact sources and detectors, free-space photon propagation may be included in the model using the first order Kirchhoff approximation to implement arbitrary boundaries (as reconstructed from the silhouettes). The resulting weight matrix is then inverted with a randomized Algebraic Reconstruction Technique (ART) algorithm. Alternatively to the diffusion equation based solutions, solutions to the radiative transfer equation may be utilized which may be more accurate at or near boundaries. However, the use of more accurate propagation models may add computational burden, and so may require highly sophisticated computer processors. Also, the diffusion approximation for in-vivo imaging has been proven effective, so either method may be employed.

To register the source positions of the sources in space, the pattern scan may be repeated on a mock diffusive layer placed in the chamber after specimen measurements are completed, and the center of the photon distribution pattern collected may be used to determine the exact location of each source. All diffuse photons propagating through tissue of the specimen or mock diffuse layers may be acquired at the emission and excitation wavelengths using charge-coupled device (CCD) hardware binning. All voxels found outside the surface may be included in the inversion but given assigned zero values. For inversion, a plurality of iterations of a randomized ART inversion algorithm may be utilized, which may result in smaller inversion times.

In the regular single spectral reconstruction algorithms, or even in the case of spectral unmixing, the data from each wavelength are reversed separately. The forward problem for each wavelength used is described by a plurality of equations in which the experimental data of either transmitted or normalized fluorescent intensity is determined by the weight matrix (which is a function of the assumed optical properties at each particular wavelength, with m×n elements) multiplied by the unknown concentration of the chromophore or the fluorophore inside the object. The weight matrix has m×n elements. The result is a vector with n source-detector pair experimental data, and the unknown concentration is discretized in m mesh points. The reconstructions may be performed with the use of a random algebraic reconstruction algorithm resulting in one concentration vector for each wavelength.

To address the requirements of multi-spectral imaging, an extra dimension may be added to the inversion problem to include the multiple wavelength data. The known spectral profile of the optical properties over the different wavelengths is taken into account for the calculation of the elements of the weight matrix. The inversion is performed simultaneously with all the wavelengths for the unknown concentration.

The random ART algorithm may be implemented in two variations. In the first one, the rows of each wavelength are separated into different classes and the ART accesses all the rows of each class in random order before proceeding to the next class. In the second scheme, all the rows are included in a single class which is accessed in random order. Both approaches result in substantially similar results. Finally, an extra dimension may be added to include the different projections, which are accessed in a sequential order outside the two access schemes described before. This algorithm marks a clear improvement over other methods in multi-spectral imaging.

Artifacts due to background fluorescence (auto-fluorescence) may appear at boundaries of the specimen. These artifacts may be removed from the image by pre-processing the data with an auto-fluorescence subtraction scheme based on the observation that the normalized fluorescence Born data from a background fluorescence distribution depend linearly on the source-detector distance. Other variables which may affect the data are the wave number at the fluorescence wavelength, a calibration constant that depends on gains and attenuation in the optical components (determined once per filter), and the unknown background fluorescence concentration multiplied by the quantum yield. All auto-fluorescence background schemes assume that there is a common background value that is subtracted. All signals that are found to conform to a background distribution of fluorescence throughout the medium may be subtracted. The subtraction scheme may correct for optical heterogeneity in the diffuse medium with the use of normalized Born data.

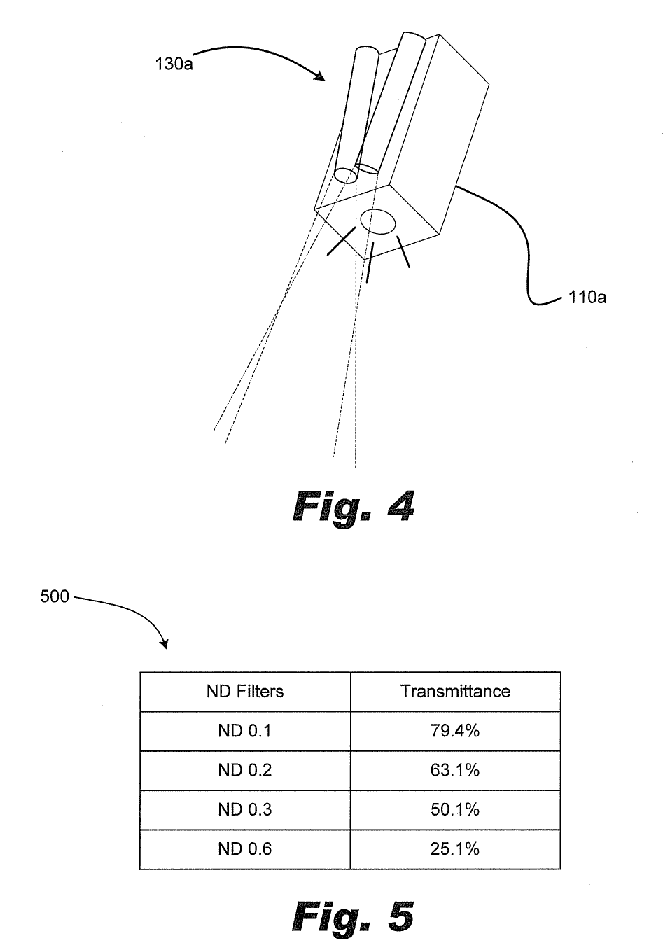

FIG. 4 illustrates a spectrum source assembly (110a) in combination with a 3D camera assembly according to an exemplary embodiment. The spectrum source assembly (110a) may comprise a projection light source. Preferably, the projection light source is an LED-based pattern projector, though the projection light source may be any type of pattern projector. An LED-based pattern projector may be preferred because it may allow for construction of a smaller DOT imaging system than systems which use other types of pattern projectors. Other projection light sources may be used which are capable of projecting a spectrum onto the specimen which may also meet desired size or power specifications. The spectrum source may also comprise a plurality of variable density filters where the projection light source is a monochromic source, wherein the filters are sequentially placed in front of the monochromic source in order to effectively create a projection equivalent to a rainbow projection. The filters may also be color spectrum filters. Alternatively, the projection light source may be capable of emitting a rainbow projection, eliminating the need for exterior filters.

The 3D camera assembly (130a) may comprise a plurality of video cameras capable of capturing a response of the specimen to the rainbow projection emitted from the spectrum source over 180 degrees. This allows the 3D camera assemblies in the imaging system to cover a full 360 degrees of the top portion of the specimen such that a three-dimensional rendering of the specimen may be adequately reconstructed in conjunction with the response to the sensor assembly.

FIG. 5 is a table (500) comprising possible transmittance values for a plurality of ND filters. The ND filter wheel (240; FIG. 2) may comprise any number of filters, each designed to pass a different intensity value. This may allow the illumination on the specimen from the illumination source assembly (120; FIG. 1) to be regulated so that the sensor, such as an NIR camera, may accurately record the response of the specimen (150; FIG. 1) to the illumination without saturating any CCD pixels in the NIR camera. Higher ND filter values block more light from the illumination source assembly than lower values. The higher value ND filters may be used when the intensity of the response is greater, and lower value ND filters may be used when the intensity of the response is less. The ND filter wheel may be controlled by the computer or other processor such that the filters may be changed automatically as the intensity of the specimen's response to the illumination changes; thus, the recorded response by the NIR camera may be more accurate. The filter wheel may comprise any filters capable of altering the intensity of the illumination onto the specimen during operation. The system may also comprise other methods of controlling the intensity of the illumination in conjunction with the ND filter wheel.

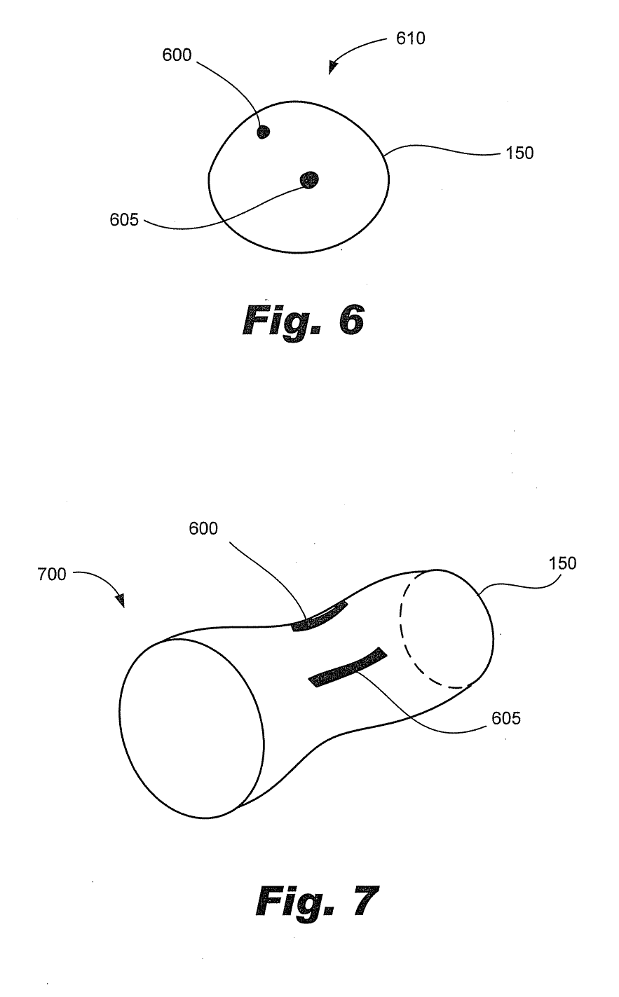

FIG. 6 shows a reconstructed axial FMT image slice (610) of a specimen (150) with first and second fluorescent tubes (600, 605) inserted into the specimen (150). FIG. 7 shows a combined three dimensional rendering (700) of the surface reconstruction together with the underlying FMT rendering of the first and second fluorescent tubes (600, 605) disposed within the specimen (150) using an exemplary embodiment of the DOT system and method of image reconstruction.

Some shape and co-registration inaccuracies may occur given the higher uncertainty of the accuracy of the FMT imaging as the illumination source penetrates deeper into the specimen. Although the size of the second tube (605) in FIGS. 6 and 7 is larger than that of the first tube (600), due to a drop in resolution as a function of depth, the sum of the reconstructed values on the area of the second tube (605) is several times higher compared to the sum of the area from the first tube (600). This ratio of the reconstructed values may be close to the original concentration ratio (in which the fluorescent dye concentration of the second tube (605) is several times higher than the fluorescent dye concentration of the first tube (600)), which demonstrates the capability of the method for quantitative measurements. Such performance can be improved with this knowledge through spatially dependent regularization.

The registration of surface and tomographic data may be generally straightforward because the surface and diffuse data may be acquired under the same geometrical frame. Simultaneous rendering allows for more accurate orientation because some high resolution anatomical information may be viewed together with fluorescence tomography data.

Motion of the specimen due to breathing, cardiac function and other physiological activity provides obstacles to in-vivo imaging because the movement causes the responses captured by both the 3D cameras and the NIR camera to differ from frame to frame, albeit slightly. The systems and method described in the present specification are nevertheless capable of providing accurate reconstructions of the specimen within acceptable resolution limits of FMT despite such motion.

A limitation of surface reconstruction from silhouettes over photo-grammetry methods is that certain concave surfaces cannot be captured. Concavities, such as joints or skin folds, with silhouette-inactive surfaces are not visible using silhouettes and as such cannot be reconstructed.

The average signal-to-noise ratio (SNR) of the system, calculated as the ratio of the mean intensity of the detected signal after background subtraction to its standard deviation, may be more than 40 or 50 decibels. The main source of noise, other than expected Poisson-distributed photonic noise and CCD read noise, may be the intensity fluctuation of the laser source. The SNR may be improved using reference fiber measurements to monitor laser fluctuations in the illumination source assembly as a function of time and accordingly correct the measurements.

A key parameter of DOT in fluorescence mode is the emission signal response as a function of light intensity and fluorophore concentration. Deviation from a linear behavior indicates photobleaching or quenching phenomena that could result in quantification errors and data misinterpretation. Photobleaching occurs when fluorescent molecules are destroyed due to the illumination, and quenching describes processes which decrease the fluorescent intensity of the fluorescent molecules. In the systems and methods described herein, no self-quenching effects or photobleaching occur while using indocyanine dye in the FMT imaging. The system is capable of clearly detecting dye concentrations of 1 nano-mol, even when using low light power. By appropriately selecting light power, sub-nano-mol concentrations may be detected for smaller volume fractions.

The preceding description has been presented only to illustrate and describe embodiments and examples of the principles described. This description is not intended to be exhaustive or to limit these principles to any precise form disclosed. Many modifications and variations are possible in light of the above teaching.

What is claimed is:

1. A multi-spectrum diffuse optical tomography imaging system for in vivo non-contact imaging, comprising:
an illumination source assembly for illuminating a specimen;
a first filter wheel adapted to control an intensity of illumination directed onto said specimen;
a three-dimensional (3D) imaging assembly for outputting an electronic (3D) model of said specimen; and
a sensor assembly for capturing a response of said specimen to said illumination source assembly and outputting corresponding tomography data;
wherein said system combines said tomography data and said 3D model for said specimen.

2. The system of claim 1, further comprising a second filter wheel configured to control a wavelength of light captured by said sensor assembly.

3. The system of claim 2, wherein said second filter wheel comprises a fluorescence filter adapted to block laser light from said illumination source assembly and allow only light of a certain wavelength to pass through.

4. The system of claim 1, wherein said sensor assembly comprises a multi-channel plate.

5. The system of claim 1, wherein said illumination source assembly comprises a plurality of lasers each outputting a beam having a different wavelength.

6. The system of claim 5, wherein said illumination source assembly comprises a beam combiner adapted to combine said beams into a multi-wavelength composite beam.

7. The system of claim 6, further comprising a linear motion stage adapted to selectively direct said composite beam onto different areas of said specimen.

8. The system of claim 1, further comprising a linear motion stage adapted to selectively direct said illumination onto different areas of said specimen.

9. The system of claim 1, wherein said 3D imaging assembly comprises two separate 3D cameras directed at opposite sides of said specimen.

10. The system of claim 9, wherein each of said 3D cameras projects a pattern of light having a spatially-varying wavelength and generates said 3D model using triangulation in which a wavelength of light in said pattern corresponds to an angle of incidence for said light on said specimen.

11. The system of claim 1, wherein 3D imaging assembly projects a pattern of light having a spatially-varying wavelength and generates said 3D model using triangulation in which a wavelength of light in said pattern corresponds to an angle of incidence for said light on said specimen.

12. The system of claim 11, wherein said 3D imaging assembly comprises a Light Emitting Diode (LED)-based pattern projector.

13. The system of claim 11, wherein said 3D imaging assembly comprises a monochromic light source and a plurality of variable density filters.

14. The system of claim 1, wherein said first filter wheel comprises a neutral density filter wheel comprising a number of different filters, each adapted to pass a different value of laser intensity and wavelength.

15. The system of claim 1, further comprising a processor-based device configured to process data acquired by said sensor assembly and said 3D imaging assembly.

16. The system of claim 15, wherein said processor-based device is further configured to control said illumination source assembly, said 3D imaging assembly and a linear motion stage adapted to direct illumination from said illumination source assembly onto different areas of the specimen.

17. A multi-spectrum diffuse optical tomography imaging system for in vivo non-contact imaging, comprising:
means for illuminating a specimen;
means for controlling an intensity of illumination directed onto said specimen;
means for sensing a response of said specimen to illumination including means for controlling a wavelength range of light reflected or transmitted by said specimen that is detected and means for outputting corresponding tomography data;
means for outputting an electronic (3D) model of said specimen;
means for combining said tomography data and said 3D model for said specimen.

18. A method of optical tomography, comprising:
illuminating a specimen;
controlling an intensity of illumination directed onto said specimen and a portion of said specimen receiving said illumination;
generating tomography data based on a response of said specimen to said illumination;
generating an electronic (3D) model of said specimen; and
combining said tomography data and said 3D model for said specimen.

19. The method of claim 18, further comprising filtering light received from said specimen by wavelength.

20. The method of claim 18, wherein said generating tomography data comprises receiving light from said specimen with a multi-channel plate.
